(19) United States
(12) Patent Application Publication
(10) Pub. No.: US 2014/0141069 A1
(43) Pub. Date: May 22, 2014

(54) BETA-GUANIDINOPROPIONIC ACID FOR THE TREATMENT OF HYPERTENSION

(71) Applicant: Lizzy Maritza Brewster, Amsterdam (NL)

(72) Inventor: Lizzy Maritza Brewster, Amsterdam (NL)

(21) Appl. No.: 14/110,520

(22) PCT Filed: Apr. 10, 2012
(86) PCT No.: PCT/NL12/50237

§ 371 Date: Dec. 27, 2013

(30) Foreign Application Priority Data
Apr. 8, 2011 (NL) PCT/NL2011/050237

(51) Int. Cl. A61K 31/197; A61K 45/06

(52) U.S. Cl. CPC A61K 31/197; A61K 45/06
USPC 424/450; 514/565; 424/474; 424/463; 514/171; 514/118

(57) ABSTRACT

The present invention relates to β-guanidinopropionic acid for the prevention and/or treatment of hypertension associated with high baseline creatine kinase levels, not necessarily associated with hyperglycemia, impaired glucose tolerance, hyperinsulinemia, insulin insensitivity, hyperamilinemia, excess adiposity or hyperlipidemia, comprising administering to a subject in need thereof a therapeutically effective dosage of a pharmaceutical composition comprising β-guanidinopropionic acid, or a pharmaceutically acceptable salt or derivative thereof, and a pharmaceutically acceptable carrier.

TECHNICAL FIELD

The present invention relates to compounds such as β-guanidinopropionic acid for use in the prevention and/or treatment of hypertension and diseases related to hypertension, preferably hypertension associated with high baseline serum levels of creatine kinase. The invention further relates to pharmaceutical compositions comprising compounds such as β-guanidinopropionic acid for use in the prevention and/or treatment of hypertension and diseases related to hypertension, wherein the hypertension is associated with high baseline serum levels of creatine kinase, and to methods for the production of said compounds for use in the prevention and/or treatment of hypertension associated with high baseline serum levels of creatine kinase and diseases related to hypertension associated with high baseline serum levels of creatine kinase.

### BACKGROUND OF THE INVENTION

The overwhelming necessity for effective and causal treatment of hypertension is well known in the art. An estimated share of more than 25% of the adult world population is affected by hypertension, which is the greatest risk factor for premature death in all industrial countries (Kearney, P M, 2005, Lancet, 365. 217-223). Even with adequate treatment, mortality is greatly increased compared to non-hypertensives, with relative risks for early mortality estimated up to threefold. Therefore, an unmet urgent need for new compounds to reduce the occurrence, consequences or complications of hypertension exists to ultimate improve public health in all countries.

Two types of hypertension are commonly distinguished in the art: essential, also referred to as primary hypertension, and secondary hypertension. Essential hypertension, for which by definition no direct cause could be identified, is by far the most prevalent hypertension type, affecting 90-95% of hypertensive patients (Carretero O A, 2000, 101 (3), 329-335). However, many factors such as obesity, smoking, stress, sedentary lifestyle, potassium deficiency (hypokalemia), sodium sensitivity, alcohol intake and vitamin D deficiency were associated with an increase in the risk of developing hypertension. In addition, risk increases with elevated levels of kidney hormone renin, aging, some inherited genetic mutations, sympathetic nervous system, overactivity and insulin resistance as well as having a family history of hypertension. Although environmental and biological circumstances are therefore known to contribute to the occurrence of essential hypertension, the pathogenesis of the condition, which occurs more frequently in men, obese people, and blacks, remains unclear and is subject of ongoing investigation.

Secondary hypertension by definition results from an identifiable cause. Therefore, in contrast to essential hypertension, treatment of the underlying cause of elevated blood pressure due to secondary hypertension is possible. Secondary hypertension may result from dysregulation of the hormone-regulating endocrine system, that regulate blood plasma volume and heart function such as Cushing's syndrome, which is a condition where the adrenal glands overproduce the hormone cortisol (Dodt C, 2009, Der Internist, 50 (1), 36-41). Furthermore, secondary hypertension might be induced by kidney disease, obesity/metabolic disorder, pre-eclampsia during pregnancy, coarctation of the aorta and certain side effects of drugs.

Though most of the mechanisms associated with secondary hypertension are generally understood, those associated with essential hypertension are far less understood. Factors, to explain so-far unexplained causes for the formation and retention of essential hypertension include the inability of the kidneys of certain individuals to excrete sodium, resulting in natriuretic factors such as Atrial Natriuretic Factor being secreted to promote salt excretion with the side effect of raising total peripheral resistance. Moreover, an overactive renin-angiotensin system is known to lead to vasoconstriction and retention of sodium and water. The resulting increase in blood volume and vasoconstriction was shown to lead to hypertension (Pimenta E, 2009, Vascular Health and Risk Management 5 (1), 453-63).

In addition, an overactive sympathetic nervous system, leading to increased stress responses and sustained endothelial damage are discussed at present.

Another new factor that was found to have an effect on blood pressure is high creatine kinase activity. The enzyme creatine kinase is known in the art to act as a physiologic energy buffer and to catalyse the reversible transfer of the phosphoryl group from phosphocreatine (PCr) to adenosine 5′-diphosphate (ADP), to generate adenosine 5′-triphosphate (ATP) and creatine (Cr), using H⁺ through the reaction:

ADP+phosphocreatine (PCr)→ATP+creatine (Cr)

Importantly, the ATP regenerating capacity of creatine kinase exceeds ATP synthesis from both oxidative phosphorylation and glycolysis. The enzyme is abundantly expressed in both the mitochondrion and the cytosol. At the mitochondrial site, it facilitates the formation of creatine phosphate, which is transported by creatine kinase to sub-cellular locations of high energy demands. At these cytosolic locations, creatine kinase is tightly bound near motor proteins involved in force generation at acto-myosin ATPase, and near ion channels, at sarcoplasmatic reticulum-Ca2⁺-ATPase and Na⁺/K⁺-ATPase where it rapidly provides ATP to these enzymes. ATP regenerated by creatine kinase is not in free equilibrium with the ATP in surrounding medium, but is micro-compartmentalized and used preferentially by ATPases involved in pressor responses. Creatine kinase fuels highly energy-demanding processes such as sodium retention, cardiovascular contractility, as well as remodelling of arteries.

The present inventor, in a previous study, assessed serum creatine kinase activity in a stratified random sample of a multi-ethnic population after the participants refrained from heavy exercise during 3 days. It is known that post exercise levels of creatine kinase are high in serum through a combination of muscle damage and temporary greater expulsion of creatine kinase from the lymphatic system to the blood stream after exercise (Brewster L M, 2006, Circulation, 114, 2034-2039). A period of 3 days substantially reduces the effect of exercise on serum creatine kinase activity. Assessed individuals were of white-European (n=503), South Asian (n=292), or African descent (n=580). Within this preceding study, high baseline creatine kinase levels were found in black men. In addition, intermediately high creatine kinase levels in South Asians, a population subgroup with blood pressure levels intermediate to the relatively low levels of white and relatively high levels of black people were found.

It was further found that the high serum creatine kinase activity after rest was due to higher creatine kinase activity in tissue and the highest creatine kinase activities were invariably seen in black people.

Furthermore, inhibiting creatine kinase activity with equimolar quantities of DNFB resulted in a mean inhibition of contraction of 0.90 in whites vs. 0.32 in blacks. Based on these results it was concluded that high vascular creatine kinase may fuel the greater contractility responses (Brewster L M, 2010, Am J Heart Circ Physiol, 299(2), 431-436).

In another setting, it was found that people with low baseline creatine kinase levels had a higher incidence of fainting, and within the group of fainters, those with recurrent fainting had the lowest creatine kinase. This finding is in line with data, pointing to lower contractility responses in those with lower creatine kinase. Therefore, it was studied whether creatine kinase activity is significantly associated with blood pressure (Brewster L M, 2006, Circulation, 114, 2034-2039). These preceding studies confirmed the relevance of creatine kinase in relation to blood pressure levels, and provided data showing that in the absence of clinical indications of muscle damage, the level of serum CK is strongly associated with blood pressure levels.

However, no approved medication or even compound in clinical testing is presently available to address essential hypertension associated with high baseline serum levels of creatine kinase. Currently, prevention of risk factors for hypertension, preferably essential hypertension, is the accepted first stage of counteracting said disease by keeping sodium/potassium balance, detection and omission of environmental toxins, monitoring changes in end/target organs (retina, kidney, heart, among others), performing lifestyle changes to achieve recommended lower blood pressure, before the initiation of prescription drug therapy is required.

Prescription drug therapy may comprise several classes of medications, collectively referred to as antihypertensive drugs, however, none of them is for causally treating essential hypertension. Compounds within a particular class, such as for example beta blockers, angiotensin II inhibitors or calcium channel blockers generally share a similar pharmacologic mechanism of action, and in many cases have an affinity for similar cellular receptors besides the group of diuretics.

The aim of medication treatment is a reduced blood pressure below 140/90 mmHg for most individuals, and lower for individuals with diabetes or kidney disease. The aim is not to counteract the pathophysiological cause for the hypertension.

Hence, a great demand exists for causal medication of essential hypertension and corresponding compounds.

The compound β-guanidinopropionic acid, is described in the prior art for treating or preventing certain metabolic disorders of human and animal metabolism, e.g. hyperglycemia, impaired glucose tolerance, hyperinsulinemia, insulin insensitivity, hyperamilinemia, excess adiposity or hyperlipidemia. In addition, it was suggested to reduce hypertension through the reduction of hyperglycemia, impaired glucose tolerance, hyperinsulinemia, insulin insensitivity, hyperamilinemia, excess adiposity or hyperlipidemia. However, it was never before suggested for use in the treatment or prevention of hypertension associated with high baseline serum levels of creatine kinase, but not necessarily associated with hyperglycemia, impaired glucose tolerance, hyperinsulinemia, insulin insensitivity, hyperamilinemia, excess adiposity or hyperlipidemia.

Recent studies demonstrate that β-guanidinopropionic acid decreases plasma glucose levels by increasing the sensitivity to insulin. This effect is based on a β-guanidinopropionic acid-induced expression of mRNA and total protein content of the insulin-responsive glucose transporter GLUT4.

β-Guanidinopropionic acid is widely used in humans as a supplement to lose weight and increase endurance, generally in a dose of between 7 and 100 mg/kg/day, but human data on its effect are lacking entirely. Numerous animal studies have been performed, mainly using doses between 1000 and 6000 milligrams/kg body weight.

Moreover, β-guanidinopropionic acid is described in the prior art to inhibit growth, transformation or metastases of mammalian cells. In particular, WO2006/034358 describes the use of creatine derivatives, including β-guanidinopropionic acid, for treating of muscle atrophy and weakness (neuromuscular disease). More specifically, it refers to the provision of methods for treatment of metabolic diseases that relate to deregulated body weight by administering to an afflicted individual a creatine derivative formulation including cyclocreatine or homocyclocreatine which modulates one or more of the structural or functional components of the creatine kinase/creatine phosphate system sufficient to prevent, reduce or ameliorate the symptoms of the disease. The effect was claimed to be prophylactic in terms of preventing or partially preventing a disease, symptom or condition thereof and/or may be therapeutic in terms of a partial or complete cure of a disease, condition, symptom or adverse effect attributed to the disease.

The use of β-guanidinopropionic acid was claimed as being especially beneficial in metabolic diseases of human and animal metabolism, e.g. associated with hyperglycemia, impaired glucose tolerance, hyperinsulinemia, insulin insensitivity, hyperamilinemia, excess adiposity or hyperlipidemia, and to reduce high blood pressure through reduction of hyperglycemia, impaired glucose tolerance, hyperinsulinemia, insulin insensitivity, hyperamilinemia, excess adiposity or hyperlipidemia. Formulations of β-guanidinopropionic acid and the like may be administered to patients having myoclonus as a symptom of epilepsy, neurodegenerative disease such as Parkinson's disease, multiple sclerosis or amyotrophic lateral sclerosis (ALS) and Tourette's syndrome.

The prior art is directed towards β-guanidinopropionic acid for use in treating a patient so as to result in any enhancement of muscle performance, building muscle tissue, treating a neuromuscular disorder, improving muscle endurance or reducing fat tissue, thereby pointing on muscle disorders. However, no guidance is so far provided as to the possible treatment of hypertension associated with high CK, and which is not necessarily associated with the metabolic disorders such as hyperglycemia, impaired glucose tolerance, hyperinsulinemia, insulin insensitivity, hyperamilinemia, excess adiposity or hyperlipidemia, by β-guanidinopropionic acid and the like.

WO2008/124151 describes the use of creatine derivatives including β-guanidinopropionic acid for use in the treatment of eye diseases. A general list of eye diseases is included and mentions “Ocular Hypertension” without referring to the use of β-guanidinopropionic acid specifically for ocular hypertension. As the person skilled in the art is aware of “intraocular pressure” differs pathophysiologically from hypertension associated with the blood circulation system in terms of the physiological mechanisms by with the pressure is built up and in terms of physiological liquid on with the pressure is exercised and which transmits the pressure. Hence, WO2008/124151 does not guide to β-guanidinopropionic acid for use in the preventing or treating hypertension.

Against this background and the fact that less than half of all hypertension patients under state-of-the-art medication reach applicable blood pressure goals, and that patients that are difficult to treat are found to have high creatine kinase levels, independent of metabolic disorders such as hyperglycemia, impaired glucose tolerance, hyperinsulinemia, insulin insensitivity, hyperamilinemia, excess adiposity or hyperlipidemia, the problem is that there remains a need for new compounds and treatment strategies in hypertension associated with high creatine kinase levels.

In aspects of the present invention it is intended that the modulation of the intravascular creatine kinase level in order to modulate platelet aggregation occurs by activating or inhibiting the intravascular creatine kinase level alone, without affecting the level of creatine or phosphocreatine (and ADP or ATP), or by affecting the level of creatine or phosphocreatine alone, without affecting the level of intravascular creatine kinase. That this would be possible was hitherto not acknowledged. Hence, treatment regimes in accordance with the present invention are aimed at modulating preferably only a single reactant component of the creatine kinase reaction as described herein.

### SUMMARY OF THE INVENTION

In the present invention, the inventor surprisingly found that β-guanidinopropionic acid is eligible for use in the prevention or treatment of hypertension, preferably essential hypertension associated with high creatine kinase levels and not necessarily associated with obesity or other metabolic abnormalities, by reducing the physiological effect of high baseline creatine kinase levels, wherein β-guanidinopropionic acid exhibits very potent anti-vascular contractility properties by lowering creatine kinase activity and, hence, is useful in treatment of hypertension. The effects of β-guanidinopropionic acid can not be explained by, for example, a reduction in weight. It was found that non-obese, Spontaneous Hypertension (SH) rats without hyperglycemia, impaired glucose tolerance, hyperinsulinemia, insulin insensitivity, hyperamilinemia, excess adiposity or hyperlipidemia, that were fed a creatine-free diet did not show blood pressure rise that is characteristic for such animals when their feed contained 0.1% β-guanidinopropionic acid.

It is known in the art that β-guanidinopropionic acid reduces creatine and phosphocreatine concentrations in several tissues. β-guanidinopropionic acid is taken up by cells via the creatine transporter a sodium symporter, and functions intracellularly as a competitive substrate to creatine kinase. However, the inventor surprisingly found that β-guanidinopropionic acid reduces creatine and phosphocreatine concentrations significantly in vascular and renal tissue, whereby vascular contractility is lowered and hypertension is treated.

The finding that β-guanidinopropionic acid lowered blood pressure in lean (non-obese) animals was unexpected. It was generally thought that inhibition of CK would cause serious potential side-effects in brain, heart and skeletal muscle, as CK is a critical enzyme in cell energy metabolism (see, for example, Clark J et al., 1994. Biochem J 300: 211-216; and Lindbom M et al., 2008. Card Fail 14:161-166.). In addition, some human trials have aimed to boost CK function by feeding creatine, which improves muscle energy metabolism and some studies suggest it also improves brain energetics (e.g. Beal M et al., 2011. Amino Acids 40:1305-1313). Enhancing CK by feeding creatine did not cause hypertension (Mihic S et al., 2000. Med Sci Sports Exerc 32: 291-296.). Thus, it was generally expected that inhibition of CK would not appear to be attractive or realistic for the treatment of, for example, hypertension.

The present inventor studied the level of creatine kinase (also referred to herein as the creatine kinase activity), and especially the effect of high levels of CK (referred to as hyperCKemia) and its relationship to blood pressure in more detail. It was known that serum ADP induces platelet aggregation. The present inventor has now discovered that ADP-induced platelet aggregation may be hampered by high serum levels of creatine kinase. Thus, the high creatine kinase activity was not only found to have an effect on blood pressure, but was also found to have a significant effect on blood clotting or coagulation through inhibition of platelet aggregation, thereby increasing bleeding risk. This important finding, that serum CK activity, even at physiological ranges, directly affects serum ADP levels, now results in the possibility to modulate ADP-induced platelet aggregation through modulation of the CK system. Individuals with increased CK, as found within the population, have a greater inhibition of ADP-dependent platelet aggregation, suggestive of lower ADP through higher CK activity. This finding can now be used to modulate platelet aggregation in subjects. For instance, in subjects with natural high physiological CK levels (such as occurring at high frequency in individuals of Sub-Saharan African descent), administration of PCr (phosphocreatine) to the blood reduces platelet aggregation, thereby increasing a bleeding risk. With increasing CK (creatine kinase) as found within the population, for example in individuals of Sub-Saharan African descent, there is greater inhibition of ADP-dependent platelet aggregation with PCr, suggestive of lower ADP through higher CK activity.

Therefore, modulating ADP-induced platelet aggregation is possible through modulation of the CK/PCr system. Inhibition of ADP-dependent platelet aggregation can be achieved by an activation of the CK/PCr system (wherein ADP is converted into ATP) and this has utility in preventing thrombosis in subjects with low CK activity. Stimulation of ADP-dependent platelet aggregation can be achieved by an inhibition of the CK/PCr system, for example with β-guanidinopropionic acid and has utility in preventing excessive bleeding or promote blood clotting in subjects with high CK activity. Thus, the present inventor has found that ADP-dependent platelet aggregation may be modulated by interfering with activity of the CK/PCr system, wherein β-guanidinopropionic acid reduces the flux trough the CK reaction system (also referred to as the CK/PCr system) by inhibiting creatine uptake and acting as competitive substrate for CK, such that an increased bleeding risk can be treated. The flux through the CK reaction system can be increased by increasing the level of CK (e.g. by hormone treatment or any other method known in the art) or by administration of PCr, to stimulate platelet aggregation in a subject with low CK activity.

Furthermore, as the effect of β-guanidinopropionic acid on hypertension includes as a major mechanism of disease, relaxation of smooth muscle, and as hypertension and asthma are strongly linked (Jacobs D R, Hypertension 2012:59:219-225), clustering with high CK levels in black people, β-guanidinopropionic acid could also be of use in reducing hypertonic smooth muscle of the bronchia of the lung, thus combatting lung disease, more specifically asthma.

In a first aspect, the present invention provides β-guanidinopropionic acid or a pharmaceutically acceptable salt thereof or a derivative thereof for use as a medicament.

In a second aspect, the present invention provides β-guanidinopropionic acid or a pharmaceutically acceptable salt thereof or a derivative thereof for use in the prevention and/or treatment of hyperCKemic hypertension, not necessarily associated with hyperglycemia, impaired glucose tolerance, hyperinsulinemia, insulin insensitivity, hyperamilinemia, excess adiposity or hyperlipidemia.

In a preferred embodiment of said aspect, the β-guanidinopropionic acid is effectively taken up by a vascular cell, or a kidney and a vascular cell.

In another preferred embodiment of said aspect, the β-guanidinopropionic acid is for use in the treatment of hypertension which is characterized by high baseline creatine kinase levels above 130 IU/L (International Units per Liter, as estimated at 37 degrees Celcius according to the procedures of the International Federation of Clinical Chemistry (Schumann G et al. Clin Chem Lab Med 2002; 40: 635-642), preferably above 170 IU/L, in adults.

In yet another preferred embodiment of said aspect, the β-guanidinopropionic acid, or the pharmaceutically acceptable salt or derivative thereof, is administered at a dose of between 0.001 to 1000 mg per kg body weight. Preferably at a daily dose in a range between 1 and 1000 mg per kg body weight and more preferably in a range between 110 and 500 mg per kg body weight, more preferred about 0.01mg to about 500mg per kg body weight per 24 hours; about 0.1 mg to about 500 mg per kg body weight per 24 hours; about 0.1 mg to about 250 mg per kg body weight per 24 hours, or about 1.0 mg to about 250 mg per kg body weight per 24 hours, more preferred between about 1 and about 15 mg per kg body weight per 24 hours, such as about 1.5 mg per kg body weight per 24 hours.

In a third aspect, the present invention provides a pharmaceutical composition comprising a therapeutically effective amount of β-guanidinopropionic acid or a pharmaceutically acceptable salt or derivative thereof, and a pharmaceutically acceptable carrier and/or an excipient.

In a preferred embodiment of said aspect, said carrier includes an adjuvant for example selected from the group consisting of builders, stabilizers, emulsifiers, dispersants, preservatives, buffers, electrolytes, tissue penetrating agents and tissue softening agents.

In another preferred embodiment of said aspect, said carrier and/or adjuvant comprises crystals, amorphous, liposomes, micelles, nanoparticles, mircoparticles, dendrimers, natural polymers, synthetic polymers, polysaccharides, lipids, deoxyribonucleic acids, ribonucleic acids, inorganic salts, organic salts, and/or surfactants.

In yet another preferred embodiment of said aspect, said composition further comprises an amount of β-guanidinopropionic acid or a pharmaceutically acceptable salt or derivative thereof of 0.1 to 100 wt %, preferably of 0.5 to 50 wt % and more preferably of 1 to 25 wt %.

In still another preferred embodiment of said aspect, said composition comprises a tablet, coated-tablet, effervescent tablet, soft-shell capsule, coated soft-shell capsule, hard-shell capsule, coated hard-shell capsule, solution, emulsion, suspension, suppository, aerosol, powder, granulate or lyophilized product as dosage form.

In a further preferred embodiment of said aspect, said composition is administrated orally, sublingually, parenterally, transcutaneously, intravenously, intramuscularly, subcutaneously or via inhalation.

In another preferred embodiment of said aspect, said composition is administered 1 time per day, 2 times per day, 3 times per day, 4 times per day, 5 times per day, 6 times per day, 1 time per week, 2 times per week, 3 times per week, 4 times per week, 5 times per week or 6 times per week.

In yet still another embodiment of said aspect, said composition comprises a further active ingredient as a hypertension medicament selected from the group consisting of alpha-adrenergic blockers, beta-adrenergic blockers, calcium channel blockers and/or diuretics, modulators of the renin-angiotensin-aldosteron system (RAS), such as ACE inhibitors, angiotensin receptor antagonists, renin blockers, angiotensinogenases, aldosterone blockers.

In a fourth aspect, the present invention provides a method for the production of the β-guanidinopropionic acid or a pharmaceutically acceptable salt thereof or a derivative thereof for use in the prevention or treatment of hypertension. The method may comprise the chemical manufacture of the β-guanidinopropionic acid or a pharmaceutically acceptable salt thereof or a derivative thereof and/or the pharmaceutical technological steps of manufacturing an appropriate dosage form the person skilled in the art is aware of such as milling, grinding, suspending, filling, compression, encapsulating and the like. The method may also comprise appropriate analytical measures such as spectroscopic analytical tools and the like to guarantee the pharmaceutical quality of the thus produced β-guanidinopropionic acid or a pharmaceutically acceptable salt thereof or a derivative thereof and/or a pharmaceutical composition comprising said β-guanidinopropionic acid or a pharmaceutically acceptable salt thereof or a derivative thereof.

In a fifth aspect, the present invention provides the use of β-guanidinopropionic acid or a pharmaceutically acceptable salt thereof or a derivative thereof, in a method for the treatment and/or prophylaxis of hypertension comprising administering to a subject in need thereof a therapeutically effective amount of the pharmaceutical composition.

In a preferred embodiment of said method for the prevention and/or treatment of hypertension, said therapeutically effective amount is in the range of 0.001 to 1000 mg per kg body weight, preferably at a daily dose in a range between 1 and 1000 mg per kg body weight and more preferably in a daily dose range between 110 and 500 mg per kg body weight.

The individual or subject in aspects of the present invention is a high CK subject, high CK subjects being defined as a subject having a CK level of more than 130 IU/L (high CK activity), or a very high CK subject with a CK level of more than 170 IU/L (very high CK activity), after three days of rest, preferably a human, more preferably a human of sub-Saharan or south-east Asian descent, more preferably a male human of sub-Saharan or south-east Asian descent and yet more preferably a male human of sub-Saharan or south-east Asian descent having high baseline creatine kinase levels.

An individual or subject in aspects of the present invention is a low CK subject, low CK subjects being defined as a subject having a CK level of less than 95 IU/L (low CK acitivity), or a very low CK subject with a CK level of less than 75 IU/L (very low CK activity), after three days of rest, preferably a human.

In another aspect, the present invention provides a method of modulating at least one blood circulation parameter selected from blood pressure and blood platelet aggregation in a subject, said method comprising interfering with the creatine kinase reaction by modulating the level of creatine, phosphocreatine and/or creatine kinase, preferably inhibition of creatine kinase, in a vascular smooth muscle cell, vascular endothelial cell or kidney cell in said subject, wherein said modulation, preferably inhibition, is achieved by administering to a subject in need thereof a therapeutically effective amount of a pharmaceutical composition as defined in any one of claims 7 to 12 or a substance that activates the creatine kinase reaction selected from an anabolic hormone including thyroxine hormone, a glucocorticoid hormone; and phosphocreatine and creatine. Preferably, in a method according to this aspect, effective modulation, preferably inhibition, of the creatine kinase reaction is indicated by a change in the level of creatine, phosphocreatine and/or creatine kinase in the serum of said subject. In an alternative preferred embodiment, said method of modulating a blood circulation parameter in a subject selected from blood pressure and blood platelet aggregation is a method for treating hypertension, coagulopathy and/or asthma in a subject with high creatine kinase levels, or a method for treating hypotension and/or thrombosis in a subject with low creatine kinase levels.

A method for treating hypertension and/or coagulopathy according to the invention may suitably comprise the inhibition of the creatine kinase reaction by administering to a subject in need thereof a therapeutically effective amount of a pharmaceutical composition as defined herein above.

A method for treating hypotension and/or thrombosis according to the invention may suitably comprise the activation of the creatine kinase reaction by administering to a subject in need thereof a therapeutically effective amount of a substance that activates the creatine kinase reaction selected from a anabolic hormone such as thyroxine hormone, a glucocorticoid hormone; creatine and phosphocreatine.

### BRIEF DESCRIPTION OF THE DRAWINGS

FIG. 1 shows the concept of β-guanidinopropionic acid (GPA) for the use in the treatment and/or prevention of hypertension exemplified by the molecular interaction in a smooth muscle cell of the vascular tissue.

FIG. 2 shows the result of a pilot experiment of blood CK titration at 1 μmol/L ADP. It is clearly shown that with increasing CK as found within the population, there is greater inhibition of ADP-dependent platelet aggregation with CrP (phosphocreatine), suggestive of lower ADP through higher CK activity.

FIG. 3 shows the systolic blood pressure of the spontaneously hypertensive rat, a high CK hypertension animal model, during 4 weeks of treatment with β-GPA. Data are means±SEM; N=8 per group; p<0.0001.

FIG. 4 shows the non-physiological vasodilation of mesenteric arteries of spontaneously hypertensive rats in vitro incubation with the CK-inhibitor dinitrofluorobenzene. Vasodilation of mesenteric arteries of rats treated with β-GPA before sacrifice was significantly increased compared to controls. Data are means±SEM; N=5 to 6 per group; p<0.01.

### DETAILED DESCRIPTION OF THE INVENTION

### DEFINITIONS

In the context of this specification, the term “β-guanidinopropionic acid”, also referred to as guanidinopropionic acid, beta-guanidinopropionic acid, β-GPA or 3-GPA includes reference to the chemical 3-(diaminomethylideneamino)propanoic acid, and pharmaceutically acceptable salts and derivatives thereof. β-guanidinopropionic acid (molecular formula C₄H₉N₃O₂) is a crystalline white powder with a melting point of 222° C. and a molecular weight of 131.13 g/mol. β-guanidinopropionic acid is soluble in water up to 50 mg/ml and gives a colourless solution. Though the oral availability of β-guanidinopropionic acid is well established, the basic uptake mechanism has not been studied extensively, although transport by the proton coupled amino acid transporter hPTAT1 has been suggested (Metzner L, ET AL. Mol Pharm. 2009; 6:1006-11).

### β-Guanidinopropionic Acid Has The Structural Formula

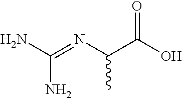

In the context of this specification, the term “derivatives” includes reference, but is not limited to, the chemicals guanidinoacetic acid (GAA) and 4-guanidinobutyric acid (4-GBA). Guanidinoacetic acid, also referred to as 2-guanidinoacetic acid, guanidinoacetic acid, N-amidinoglycine or N-guanylglycine has a molecular formula C₃H₇N₃O₂ and a molecular weight of 117.11 g/mol. Guanidinoacetic acid has the structural formula:

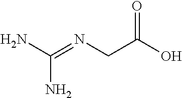

4-guanidinoacetic acid has the molecular formula C₅H₁₁N₃O₂, the molecular weight 145.16 g/mol and the structural formula:

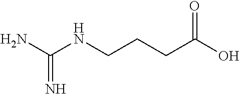

In the context of this specification, “pharmaceutically acceptable salts” include, but are not limited to, those formed from: acetic, ascorbic, aspartic, benzoic, benzenesulfonic, citric, cinnamic, ethanesulfonic, fumaric, glutamic, glutaric, gluconic, hydrochloric, hydrobromic, lactic, maleic, malic, methanesulfonic, naphthoic, hydroxynaphthoic, naphthalenesulfonic, naphthalenedisulfonic, naphthaleneacrylic, oleic, oxalic, oxaloacetic, phosphoric, pyruvic, p-toluenesulfonic, tartaric, trifluoroacetic, triphenylacetic, tricarballylic, salicylic, sulfuric, sufamic, sulfanilic and succinic acid.

The term “creatine kinase” abbreviated CK, as used herein, refers to the enzyme also known as creatine phosphokinase (CPK) or phospho-creatine kinase (EC 2.7.3.2) that catalyses the conversion of creatine and consumes adenosine triphosphate (ATP) to create phosphocreatine and adenosine diphosphate (ADP). The CK enzyme reaction is reversible, such that also ATP can be generated from phosphocreatine and ADP.

In the context of this specification, a “condition associated with high baseline creatine kinase levels” refers to any clinical condition characterised by or otherwise involving an increased baseline creatine kinase level relative to a normal reference level. Conditions associated with high baseline creatine kinase levels include, but are not limited to: certain metabolic disorders of human and animal metabolism, e.g. hyperglycemia, impaired glucose tolerance, hyperinsulinemia, insuline insensitivity, hyperamilinemia, excess adiposity or hyperlipidemia. In particular, and in preferred embodiments, high baseline creatine kinase levels are associated with hypertension and/or coagulopathy such as an increased bleeding tendency and/or asthma.

In the context of this specification, a “condition associated with low baseline creatine kinase levels” refers to any clinical condition characterised by or otherwise involving a decreased baseline creatine kinase level relative to a normal reference level. Conditions associated with low baseline creatine kinase levels include, but are not limited to hypotension and/or thrombosis.

In the context of the specification, the term “high baseline creatine kinase levels” refers to the prevalence of elevated creatine kinase activity in the tissue of interest, preferably in tissue related to the blood circulation system, more preferably in the vascular tissue or in kidney tissue, most preferably the level of intravascular creatine kinase. Creatine kinase activity is determined from reasonably accessible blood serum of an individual as a good estimation of tissue creatine kinase activity, wherein “high” activity is significantly above the average value for that respective group of individuals, e.g. for the individuals of one gender or of one ethnic group and the like. Preferably, high versus low creatine kinase activity is defined as the highest 25% (>170 IU/L), respectively the lowest 25% (<75 IU/L) creatine kinase activity in the general population after 3 days of rest, in otherwise healthy people, without clinical or laboratory evidence of muscle damage. There is evidence to include>130 IU/L as high CK activity. As creatine levels depend on sex and ethnicity, high serum CK levels are differently defined for sex-ethnic groups, as median levels for white women, white men, black women and black men are 72, 110, 124 and 213 IU/L respectively.

In the context of this specification, the terms “treatment” and “treating” refer to any and all uses which remedy a condition or disease or symptoms thereof, prevent the establishment of a condition or disease or symptoms thereof, or otherwise prevent or hinder or reverse the progression of a condition or disease or other undesirable symptoms in any way whatsoever.

In the context of this specification, the term “therapeutically effective amount” includes within its meaning a non-toxic amount of β-guanidinopropionic acid sufficient to provide the desired therapeutic effect. The exact amount will vary amongst others from subject to subject depending on the age of the subject, the gender, the ethnic origin, their general health, the severity of the disorder being treated and the mode of administration. It is therefore not possible to specify an exact “therapeutically effective amount”. However one skilled in the art would be capable of determining a “therapeutically effective amount” by routine trial and experimentation.

In the context of this specification, the term “hypertension” is directed to elevated blood pressure, characterized by 140 and 90 mmHg as systolic and diastolic average blood pressure value, respectively, in blood vessels which are formed by vascular tissue in general, and by endothelium, connective tissue and smooth muscle tissue in particular. Preferably, said term is directed to essential hypertension. More preferably, said term is directed to essential arterial and/or essential pulmonary hypertension.

The invention also envisions the preventive treatment of subjects with prehypertension, which term refers to subjects having blood pressures between 130-140 and 80-90 mmHg. For ease of terminology, prehypertension is included in the term hypertension, unless otherwise defined herein.

In the context of this specification, the term “diseases caused by hypertension” is directed to such pathophysiological conditions that causally result from the prevalence of the pathophysiological condition of hypertension.

In the context of this specification, the term “anti-hypertonic” is defined to mean an amount of β-guanidinopropionic acid that is capable to significantly reduce the systolic and/or diastolic blood pressure (SBP, DBP, respectively) of a person with elevated blood pressure and high CK, but not necessarily associated with hyperglycemia, impaired glucose tolerance, hyperinsulinemia, insulin insensitivity, hyperamilinemia, excess adiposity or hyperlipidemia.

The term “anti-hypertonic agent” refers to a chemical compound useful in the treatment of hypertension characterized by elevated blood pressure.

The term “coagulopathy”, as used herein, refers to blood clotting disorders and bleeding disorders, in particular defects in the mechanism for blood clotting based on ADP-induced platelet aggregation, such as inhibition of ADP-induced platelet aggregation associated with high CK.

The term “hypotension”, as used herein, refers to abnormally low blood pressure and is the opposite of hypertension, which is high blood pressure. Hypotension is generally considered as systolic blood pressure less than 90 millimeters of mercury (mm Hg) or diastolic less than 60 mm Hg.

The term “thrombosis”, as used herein, refers to the formation of a blood clot or thrombus inside a blood vessel, obstructing the flow of blood through the circulatory system.

The term “asthma”, as used herein, refers to the common chronic disease of the airways characterized by variable and recurring airflow obstruction and (smooth muscle) bronchospasm.

The term “carrier” refers to a pharmaceutically acceptable means of formulating the active ingredient to allow the active ingredient to perform its pharmacological action on the desired physiological site.

The term “adjuvant” refers to a pharmaceutically acceptable means of actively or passively enhancing the active ingredient's interaction with the desired physiological target.

### PREFERRED EMBODIMENTS

The anti-hypertension activity of β-guanidinopropionic acid in individuals with high CK not necessarily associated with hyperglycemia, impaired glucose tolerance, hyperinsulinemia, insulin insensitivity, hyperamilinemia, excess adiposity or hyperlipidemia, was not previously reported in prior art.

Surprisingly, β-guanidinopropionic acid showed an anti-hypertension effect in hyperCKemic rats without hyperglycemia, impaired glucose tolerance, hyperinsulinemia, insulin insensitivity, hyperamilinemia, excess adiposity or hyperlipidemia, in addition to those physiologic effects already described in prior art for that compound. Therefore, the invention relates to β-guanidinopropionic acid or a pharmaceutically acceptable salt thereof or a derivative thereof for use in the prevention or treatment of hypertension associated with hyperCKemia in an individual, preferably in a mammal, more preferably in a human, dog, cat, horse, cow, pig, sheep, goat, mouse, rat, gunny pig, elephant, camel, giraffe, hippopotamus and the like, most preferably in a human.

The invention further relates to a method for the prevention (prophylaxis) or treatment of hypertension in a subject in need thereof with high CK comprising administration to the subject of a therapeutically effective amount of β-guanidinopropionic acid, in an amount that inhibits or reduces the CK reaction, as defined above. Upon administration the β-guanidinopropionic acid is taken up by a cell in the vascular wall, such as by an endothelium or smooth muscle cell, or by a kidney cell and a cell in the vascular wall. The action of the β-guanidinopropionic acid is preferably to reduce vascular contractility, wherein the β-guanidinopropionic acid is preferably inhibiting the CK pathway by reducing the intracellular phosphocreatine and creatine concentration, wherein the creatine kinase reaction in the vascular muscle or epidermal cells or kidney cells is preferably effectively competitively reduced or inhibited, and wherein the creatine kinase reaction in the heart muscle cells (e.g. the cardiac function) is preferably unaffected.

The invention further relates to a method for the prevention (prophylaxis) or treatment of coagulopathy in a subject in need thereof comprising administration to the subject of a therapeutically effective amount of β-guanidinopropionic acid, in an amount that inhibits or reduces the CK reaction, as defined above, to reduce bleeding tendency.

The invention further relates to a method for the prevention (prophylaxis) or treatment of hypotension in a subject in need thereof comprising administration to the subject of a therapeutically effective amount of a substance that activates the CK reaction, such as an anabolic hormone including thyroxine or glucocorticoid hormone or phosphocreatine or creatine, in an amount that activates the CK reaction, as defined above.

The invention further relates to a method for the prevention (prophylaxis) or treatment of thrombosis in a subject in need thereof comprising administration to the subject of a therapeutically effective amount of a substance that activates the CK reaction, such as an anabolic hormone including thyroxine or glucocorticoid hormone or phosphocreatine or creatine, in an amount that activates the CK reaction, as defined above.

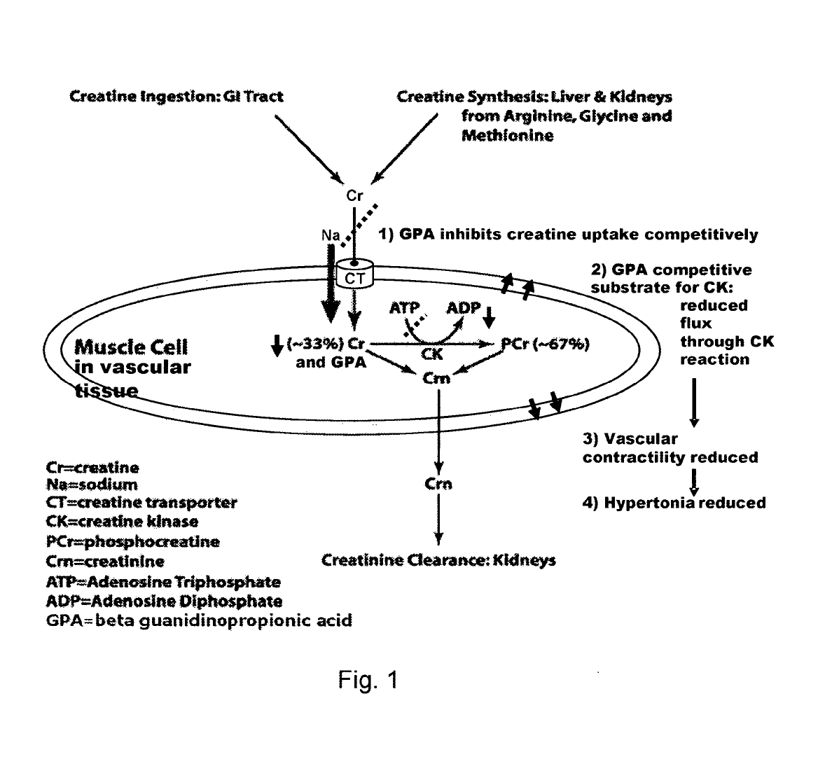

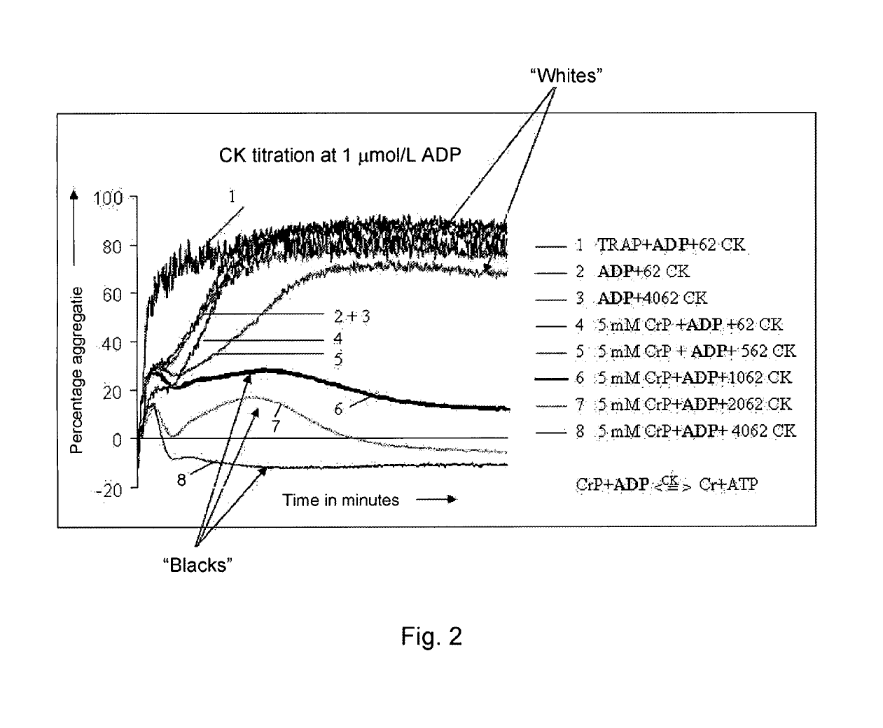

Without wishing to be bound by theory, it is believed that β-guanidinopropionic acid inhibits the flux through the creatine kinase reaction, by competitively inhibiting cellular creatine uptake (FIG. 1). Further upon uptake, β-guanidinopropionic acid is phosphorylated in cytoplasm, but both β-guanidinopropionic acid and phosphorylated β-guanidinopropionic acid are “inefficient substrates” for the creatine kinase reaction and therefore competitively inhibit the creatine kinase to interact with the genuine substrate creatine: β-guanidinopropionic acid's in vitro Vmax values are <1% of the Vmax values of creatine and phosphocreatine (FIG. 1). High doses of β-guanidinopropionic acid lead the body respond to the chronic energy depletion, by increasing glucose uptake, reducing fat stores and converting Type IIb glycolytic, high CK muscle fibers (fast-twitch fibers) into Type 1, low CK, oxidative fibers (slow-twitch or fatigue resistant fibers). Furthermore, high doses of β-guanidinopropionic acid may reduce myocardial phosphocreatine and creatine concentrations by up to 80%. Although this leads to measurable alterations in cellular energy metabolism, with 1% β-guanidinopropionic acid in rat chow, up to 8 weeks of feeding in rats does not lead to reduced cardiac function or overtly reduced sprint capacity in vivo. Hence, proper dosing of β-guanidinopropionic acid can be achieved such that heart muscle system is unaffected, whereas the creatine kinase reaction system in other tissues will be sufficiently modulated to achieve the desired anti-hypertonic therapeutic effect.

Skeletal muscles feature the highest known creatine kinase activity. Cardiac creatine kinase activity is around a factor of 2.5 to 5 lower than skeletal muscle creatine kinase activity and brain creatine kinase activity a factor of 5 to 10 lower. It is of considerable importance that vascular and kidney creatine kinase activity are around a factor of 50 to >100 lower than skeletal muscle. Therefore low-dose β-guanidinopropionic acid has differential effects on the different tissues, and smooth vascular muscle and kidney tissue are a more sensitive target. Consequently, by adopting the dose to a preferably low regimen as set out in the present invention, a selective creatine kinase activity reduction can be triggered. Hence, a preferred embodiment of this invention comprises a selective targeting pharmacological strategy. In consequence, side effects which would be associated with an undue creatine kinase activity reduction in skeletal and heart tissue are significantly reduced or diminished. Hence, the present invention provides a novel safe and efficient way of treating and preventing hypertension associated with high baseline creatine activity levels. That CK inhibition provides vasodilation was established by experimental evidence.

The hypertension may be selected from the group consisting of: arterial hypertension and pulmonary hypertension.

Pharmaceutical compositions include those suitable for oral, parenteral (including subcutaneous, intradermal, intramuscular, intravenous and intraarticular), inhalation (including use of metered dose pressurised aerosols, nebulisers or insufflators, and intranasal dosing systems), rectal and topical (including dermal, buccal, sublingual and intraocular) administration.

The compositions may conveniently be presented in unit dosage form and may be prepared by any of the methods well known in the art of pharmacy. All methods include the step of bringing β-guanidinopropionic acid as defined herein into association with a carrier which constitutes one or more accessory ingredients. In general, the compositions are prepared by uniformly and intimately bringing into association the β-guanidinopropionic acid with a solid carrier or finely divided solid carrier, or both and then, if necessary, shaping the product into the desired composition.

Generally, an effective dosage of β-guanidinopropionic acid present in pharmaceutical and other compositions of the present invention is expected to be in the range of about 0.001 mg to about 1000 mg per kg body weight per 24 hours; about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours, or about 1.0 mg to about 250 mg per kg body weight per 24 hours. More typically, an effective dose range is expected to be in the range of about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours, such as about 1.5 mg per kg body weight per 24 hours; about 5.0 mg to about 50 mg per kg body weight per 24 hours; about 5.0 mg to about 20 mg per kg body weight per 24 hours, or about 5.0 mg to about 15 mg per kg body weight per 24 hours. Generally, an amount of 0.1-3 wt. % as part of the daily food intake is envisioned in the present invention, and treatment can be dosed depending on the desired therapeutic result.

Compositions comprising β-guanidinopropionic acid or a pharmaceutically acceptable salt or derivative thereof may contain an amount of said β-guanidinopropionic acid of 0.1 to 100 wt %, preferably 0.5 to 50 wt %, more preferably 1 to 25 wt % and still more preferably 1 to 10 wt %.

Compositions suitable for buccal (sublingual) administration include lozenges comprising β-guanidinopropionic acid in a flavoured base, usually sucrose and acacia or tragacanth; and pastilles comprising β-guanidinopropionic acid in an inert base such as gelatine and glycerin or sucrose and acacia.

Compositions comprising β-guanidinopropionic acid suitable for oral administration may be presented as discrete solid dosage forms such as gelatine or HPMC capsules, cachets or compressed tablets, each containing a predetermined amount of β-guanidinopropionic acid, as a powder, granules, as a solution or a suspension in an aqueous liquid or a non-aqueous liquid, or as an oil-in-water liquid emulsion or a water-in-oil liquid emulsion. β-guanidinopropionic acid may also be present in a paste.

When the compositions comprising β-guanidinopropionic acid are formulated as capsules, the β-guanidinopropionic acid may be formulated with one or more pharmaceutically acceptable carriers such as starch, lactose, microcrystalline cellulose, silicon dioxide and/or a cyclic oligosaccaride such as cyclodextrin. Additional ingredients may include lubricants such as magnesium stearate and/or calcium stearate. Suitable cyclodextrins include α-cyclodextrin, β-cyclodextrin, γ-cyclodextrin, 2-hydroxyethyl-β-cyclodextrin, 2-hydroxypropyl-cyclodextrin, 3-hydroxypropyl-β-cyclodextrin and tri-methyl-β-cyclodextrin. The cyclodextrin may be hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Tablets may be prepared by compression or moulding, optionally with one or more accessory ingredients. Compressed tablets may be prepared by compressing in a suitable pelletizing machine β-guanidinopropionic acid in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered β-guanidinopropionic acid moistened with an inert liquid diluent, in a suitable machine. The tablets may optionally be coated, for example, with an enteric coating and may be formulated so as to provide slow or controlled release of β-guanidinopropionic acid therein.

Compositions for parenteral administration include aqueous and non-aqueous sterile injectable solutions or nanosuspensions which may contain anti-oxidants, buffers, bacteriostats and solutes which render the formulation isotonic with the blood of the intended recipient, and which may include suspending agents and thickening agents. A parenteral composition may comprise a cyclic oligosaccharide such as hydroxypropyl-β-cyclodextrin. The compositions may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use.

Compositions suitable for transdermal administration may be presented as discrete patches adapted to remain in intimate contact with the epidermis of the recipient for a prolonged period of time. Such patches suitably comprise β-guanidinopropionic acid as an optionally buffered aqueous solution of, for example, 0.01 M to 10 M, more preferably 0.05 to 1 M, even more preferably 0.1 M to 0.2 M concentration with respect to the compound or as a paste or suspension. Such patches may liberate the contained pharmaceutical preparation from the reservoir membrane- or matrix-controlled.

Aerosol compositions for delivery to the lung by inhalation may, for example be formulated as solutions or suspensions, preferably aqueous suspensions or solutions and/or suspensions in liquefied propellant delivered from pressurised packs, such as a metered dose inhaler, with the use of a suitable liquefied propellant. Suitable propellants include a fluorocarbons or a hydrogen-containing fluorocarbon or mixtures thereof, particularly hydrofluoroalkanes. The aerosol composition may be excipient free or may optionally contain additional composition excipients well known in the art, such as surfactants e.g. oleic acid or lecithin and/or cosolvents e.g. ethanol. Pressurised compositions will generally be retained in a canister (e.g. an aluminium canister) closed with a valve (e.g. a metering valve) and fitted into an actuator provided with a mouthpiece.

Alternatively, a dry powder preparation of β-guanidinopropionic acid can be used to administer said formulation as a dry powder inhaler without propellants and a related suspension formulation.

Medicaments for administration by inhalation desirably have a controlled particle size. The optimum particle size for inhalation into the bronchial system is usually 1-10 μm, preferably 1-5 μm to target the alveolar sacs. Particles having a size above 20 μm are generally too large when inhaled to reach the small airways. When the excipient is lactose it will typically be present as milled lactose, wherein not more than 85% of lactose particles will have a mass mean diameter (MMD) of 60-90 gm and not less than 15% will have a MMD of less than 15 μm.

Compositions for rectal administration may be presented as a suppository with lipophilic carriers such as half-synthetic blends of mono-, di- and triglycerides as well as natural cocoa butter or hydrophilic polyethylene glycol, or as an enema wherein the carrier is an isotonic liquid such as saline. Additional components of the compositions may include a cyclic oligosaccharide, for example, a cyclodextrin, as described above, such as hydroxypropyl-β-cyclodextrin, one or more surfactants, buffer salts or acid or alkali to adjust the pH, isotonicity adjusting agents and/or anti-oxidants.

The composition may also be administered or delivered to target cells in the form of liposomes. Liposomes are generally derived from phospholipids or other lipid substances and are formed by mono- or multi-lamellar hydrated liquid crystals that are dispersed in an aqueous medium. Specific examples of liposomes that may be used to administer or deliver a compound formula (I) include synthetic cholesterol, 1,2-distearoyl-sN-glycero-3-phosphocholine, 3-N-[(-methoxy poly(ethylene glycol)2000)carbamoyl]-1,2-dimyrestyloxy-propylamine (PEG-cDMA) and 1,2-di-o-octadecenyl-3-(N,N-dimethyl)aminopropane (DODMA). These liposomes may hold the hydrophilic active ingredient in the core or integrate it into PEG chains within the layer(s).

The compositions may also be administered or delivered in the form of nanoparticles. Biodegradable nanoparticles formed from synthetic polymers such polycyanoacrylate or from natural polymers such as albumin or gelatin were shown to aid in drug targeting, thereby reducing the amount of required dosage and side effects (Fuchs, S., 2010, J Drug Del Sc, 20(5) 331-342)

The compositions may also be administered in the form of microparticles. Biodegradable microparticles formed from polylactide (PLA), polylactide-co-glycolide (PLGA), and ε caprolactone have been extensively used as drug carriers to increase plasma half life and thereby prolong efficacy (R. Kumar, M., 2000, J Pharm Pharmaceut Sci., 3(2) 234-258).

The compositions may incorporate a controlled release matrix that is composed of sucrose acetate isobutyrate (SAIB) and organic solvent or organic solvent mixtures. Polymer additives may be added to the vehicle as a release modifier to further increase the viscosity and slow down the release rate. Xanthanodien may be added to the SAIB delivery vehicle to form SAIB solution or suspension compositions. When the formulation is injected subcutaneously, the solvent diffuses from the matrix allowing the SAIB-drug or SAIB-drug-polymer mixtures to set up as an in situ forming depot.

The co-administration of β-guanidinopropionic acid or a pharmaceutically acceptable salt thereof or a derivative thereof and one or more further anti-hypertension agents may be simultaneous or sequential. Simultaneous administration may be effected by β-guanidinopropionic acid being in the same unit dose as an anti-hypertension agent, or β-guanidinopropionic acid and the anti-hypertension agent may be present in individual and discrete unit doses administered at the same, or at a similar time. Sequential administration may be in any order as required. The anti-hypertension agent may be selected from the group consisting of ACE inhibitors, alpha blockers, angiotensin II receptor antagonists, beta blockers, calcium channel blockers and/or diuretics.

The inventor found previously that the efficacy of beta-blockers in reducing systolic blood pressure and the efficacy of angiotensin-converting enzyme inhibitors in achieving diastolic blood pressure goals did not significantly differ from placebo (weighted mean difference −3.53 mm Hg [95% CI, −7.51 to 0.45 mm Hg]; relative risk for angiotensin converting enzyme inhibitors, 1.35[CI, 0.81 to 2.26]). Furthermore, calcium blockers and diuretics led to the greatest responses, but only calcium channel blockers remained effective in patients with a baseline diastolic blood pressure of 110 mm Hg or greater (Brewster L M, 2004, Ann Intern Med, 141, 614-627). It was therefore concluded that drugs that indirectly counteract the effect of creatine kinase at the contractile proteins (sarcoplasmatic reticulum-Ca²⁺-ATPase and acto-myosin ATPase), and in the kidney (Na⁺/K⁺-ATPase) are most effective in patients with high baseline creatine kinase levels.

These ATPases play an important role in the vascular muscle cells and kidney cells. In particular, inhibition of CK in the kidney cells is believed to reduce salt (sodium) uptake and hence facilitates salt excretion. This is an aspect of the anti-hypertonic nature of β-guanidinopropionic acid as envisioned herein. Therefore, in the context of this invention, the further anti-hypertension agent to be combined with β-guanidinopropionic acid (which is directly counteracting the effect of creatine kinase) preferably to achieve a synergistic effect in the treatment of hypertension, is preferably selected from the group consisting of captopril, zofenopril, enalapril, ramipril, quinapril, perindopril, lisinopril, benazepril, fosinopril, verapamil, gallopamil, diltiazem, amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, hydrochlorothiazide, furosemide, ethacrynic acid, torsemide, bumetanide. acetazolamide, methazolamide spironolactone, amiloride and triamterene.

In the context of this invention, a new pathophysiological concept was translated into new modalities for hypertension treatment with considerable low side effects as this substance, that naturally occurs in the body and which lacks any ring structure, avoids xenobiotic metabolism in contrast to virtually all hypertensive drugs.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be further described in greater detail by reference to the following specific examples, which should not be construed as in any way limiting the scope of the invention.

### EXAMPLES

### Example 1

### A Prospective Cohort Study To Clarify Whether Normotensive Subjects With High Baseline Creatine Kinase Activity (CK) In The Population Have A Greater Risk To Develop Hypertension Than Subjects With Low Baseline Creatine Kinase Activity

### Materials and Methods

In a longitudinal cohort study, a random population sample (n=1441) is reassessed 10 years after an initial study whether normotensive subjects with high CK have developed higher blood pressures after 10 years than normotensives with low CK baseline. Therein, high vs. low CK is defined as previously mentioned. Serum CK in the highest vs. the lowest tertile in the general population after 3 days of rest, in otherwise healthy people was determined.

Sample size calculation was based on earlier retrospective studies, including 162 normotensives at baseline in the groups with high vs. low CK. Clinical Studies Medical history included self-identified ethnicity, physical examination including weight and height, an electrocardiogram, and parameters of systemic cardiovascular hemodynamics: supine and sitting resting blood pressure with an oscillometric device with an adjusted cuff size on the non-dominant arm, at heart level; heart rate, cardiac output and total peripheral resistance (standing and supine), using a Bmeye Nexfin blood-pressure monitor for continuous non-invasive finger arterial blood pressure measurement.

Central blood pressure and pulse wave velocity were assessed by a Arteriograph device. Laboratory studies included resting serum CK after 3 days of rest, shown to be feasible, and CK isoenzymes, glucose, creatinine, liver enzymes (ASAT, ALAT, gamma GT), TSH (subclinical hypothyroidism as a cause of high CK), sodium, potassium, calcium, magnesium, and fasting glucose. All procedures are running and available.

### Result

Normotensive subjects with a high baseline CK are at greater risk to develop hypertension. It was shown that the relative risk of hypertension or prehypertension (>129 mm Hg systolic and >79 diastolic) in those with normal blood pressure with a low baseline in CK is lower than those in high groups, adjusted for sex, BMI, ethnicity and other significant predictors of blood pressure (at p=0.10)

### Example 2

### The Association Between CK And Blood Pressure Are Replicated In A Large, Multiethnic, Population Study

### Methods

A prospective cohort study assessing general health and cardiovascular in a random population sample on 60.000 people (10.000 of each of these ethno-cultural backgrounds: white, Moroccan, South-Asian, black African, Black-Caribbean, Turkish) is performed.

The association between CK and peripheral arterial systolic and diastolic blood pressure in a large, multiethnic population, independent from sex, BMI, and ethnicity, and other significant predictors of blood pressure (p=0.10) were evaluated. The independent association of CK with central blood pressure, pulse wave velocity, and peripheral resistance was assessed.

The clinical studies were identical to those conducted in Example 1.

### Result

The causal association between CK and blood pressure is not refuted.

### Example 3

### Studying A Fine-Tuned Attenuation Of The Flux Through The CK Reaction By Assessing Low Dose Administration β-Guanidinopropionic Acid To Treat Or Prevent Hypertension

If creatine kinase causes hypertension, the human body will respond, haemodynamically and otherwise, to a fine-tuned attenuation of the flux through the CK reaction. Because of findings that high CK precedes hypertension in animal models, lowering blood pressure lowers CK in animal models and CK inhibition in animals is reported to lower blood pressure and the over the counter availability of beta β-guanidinopropionic acid, a competitive inhibitor of creatine uptake for human use, the inventor tested whether β-guanidinopropionic acid reduces blood pressure in humans.

### Materials and Methods

In a step-wise approach, formulation (preliminary tests of β-guanidinopropionic acid marketed for human use passed certified toxicological testing), pharmacokinetic and pharmacodynamic analyses including blood pressure lowering was performed, with validated challenge tests to predict the optimal pharmacologically active dose, to be used. β-guanidinopropionic acid dosing per day and individual was between 10 mg/kg and 100 mg/kg due to the fact that β-guanidinopropionic acid is well absorbed orally in animals, and a membrane transporter has been identified.

Assessments of bioavailability data of β-guanidinopropionic acid included laboratory studies as described in Examples 1 and 2, adding serum β-guanidinopropionic acid, creatine, and urine creatine/creatinine ratio, non-invasive peripheral and central haemodynamics adding venous occlusion forearm plethysmography, and, as the gold standard to assess blood pressure reduction, 24 h blood pressure measurements. Further specific tests included cardiac imaging (Echocardiography) and skeletal muscle 1H (creatine) and 31P (PCr/ATP ratio) spectroscopy.

Initially, the pharmacokinetic of β-guanidinopropionic acid and creatine after an oral load of β-guanidinopropionic acid in healthy volunteers was assessed.

Second, after uneventful pharmacogenetic data gathering, basic pharmacodynamic studies in healthy were performed in normotensive men, assessing the uptake and model the uptake and effects in muscle by in vivo 1H and 31P magnetic resonance spectroscopic imaging for respectively creatine and high energy-phosphates; as well as muscle needle biopsy for fiber type distribution (a shift from type IIb, high CK fast glycolytic fibers to type 1, low CK fibers with the use of β-guanidinopropionic acid).

Furthermore, sitting, supine and mean 24 h blood pressure levels and central and peripheral haemodynamics, blood glucose, lipids were assessed and body weight was monitored.

Finally, with successful previous stages the effect of β-guanidinopropionic acid on blood pressure in untreated stage I hypertensives (systolic blood pressure 140-159; diastolic 90-99) at different doses was studied. The tested range of doses (10 to 100 mg/kg/day) was a small fraction of the highest dose animal testing. By this low dose of inhibitor, it was possible to target low CK tissues such as kidney and vascular tissue, without affecting high CK tissue such as heart and skeletal muscle as assessed by 1H and 31P MRS, echocardiography, cardiac output with Bmeye Nexfin blood-pressure monitor for continuous non-invasive heamodynamic monitoring, and anaerobic an aerobic skeletal muscle function tests.

For a pre-defined set of doses and a binary response, CRM estimated MTD as the dose level that yields a particular target proportion of responses. The method assumes that the probabilities of both efficacy and unwanted effects increase with increasing dose. The method also provides that side effects can be defined as a binary outcome. The “acceptable” side effect level is explicitly defined and the MTD is the highest (most efficacious) dose with acceptable side effects. Main strengths of CRM are that it is less likely that subjects are treated at too high doses, more likely that they are treated with efficacious doses, and it can more accurately estimate the MTD and dose-response curve. Main disadvantages are mathematical and statistical complexities including simulation, the large dose escalations that may occur based on little information, and the dosing of first subjects at level deemed appropriate by a priori curve may be worrisome due to uncertainty surrounding this curve.

Therefore, this method was adjusted, and it was always started at the lowest dose level under consideration, enrolling 2-3 subjects in each cohort, and proceed as a standard dose escalation design in the absence of dose-limiting effects as defined above, while care was being given that any given dose escalation cannot increase by more than one level.

Blood pressure measurements beside the PK/PD studies and modelling of skeletal muscle metabolism of β-guanidinopropionic acid were performed, clinical studies include medical history, self-identified ethnicity, physical examination including weight and height, an electrocardiogram, and parameters of systemic cardiovascular hemodynamics: supine and sitting resting blood pressure with an oscillometric device; heart rate, cardiac output and total peripheral resistance (standing and supine), using a Bmeye Nexfin blood-pressure monitor for continuous non-invasive finger arterial blood pressure measurement, with an adjusted cuff size on the non-dominant arm, at heart level. Central blood pressure and pulse wave velocity were assessed with the arteriograph device. 24 h ambulatory blood pressure measurements were performed to assess day time and night time blood pressure, as a more precise measure of blood pressure regulation.

Laboratory studies included resting serum CK and isoenzymes, glucose, creatinine, liver enzymes (ASAT, ALAT, gamma GT), TSH (subclinical hypothyroidism as a cause of high CK), sodium, potassium, calcium, magnesium, and fasting glucose.

### Results

The tested range of β-guanidinopropionic acid doses (10 to 100 mg/kg/day) was a small fraction of the highest dose animal testing. By this low dose low dose of CK inhibitor, it was possible to target low CK tissues such as kidney and vascular tissue, without affecting high CK tissue such as heart and skeletal muscle.

### Example 4

### Additional Experiments

A total of 24 SH adult rats are divided into 4 groups: the test group is given 3% β-GPA , a second group is given 3% creatine, a third group is given 3% creatine +3% β-GPA; and a control group receives only normal feed. SH rats are usually also associated with high creatine kinase levels, although it is unknown whether this is cause or result of the spontaneous high blood pressure. The present inventor has now found that the high creatine kinase is the cause of the high blood pressure.

Blood pressure was measured at the tail using standard methods (tailcuff method). In the control group, the blood pressure rises. An absence in the rise of blood pressure or even a reduction in the blood pressure in the test group indicates the blood pressure lowering effect of β-GPA. In order to exclude a possible effect of creatine deficiency or a possible effect of creatine itself on the blood pressure level, the two additional control groups (3% creatine or 3% creatine +3% β-GPA). The blood pressure level in the treated group is lower than that of the control SH rats.

### Example 5

### Further Experiments

Male, non-obese, normoglycaemic, 16-weeks-old SHR without lipid abnormalities were randomly assigned to a diet with β-GPA versus a standard diet without β-GPA (N=8 per group). Blood pressure was measured weekly with the tail cuff method. All blood pressure measurements were performed unblinded under equal conditions. Body temperature was kept constant during the measurements at 36-38° C. Also tested was the effect of β-GPA and the CK-inhibitor dinitrofluorobenzene (DNFB) on vasodilation of SHR mesenteric arteries in vitro in a wire myograph. Data are expressed as mean (SEM).

### Results

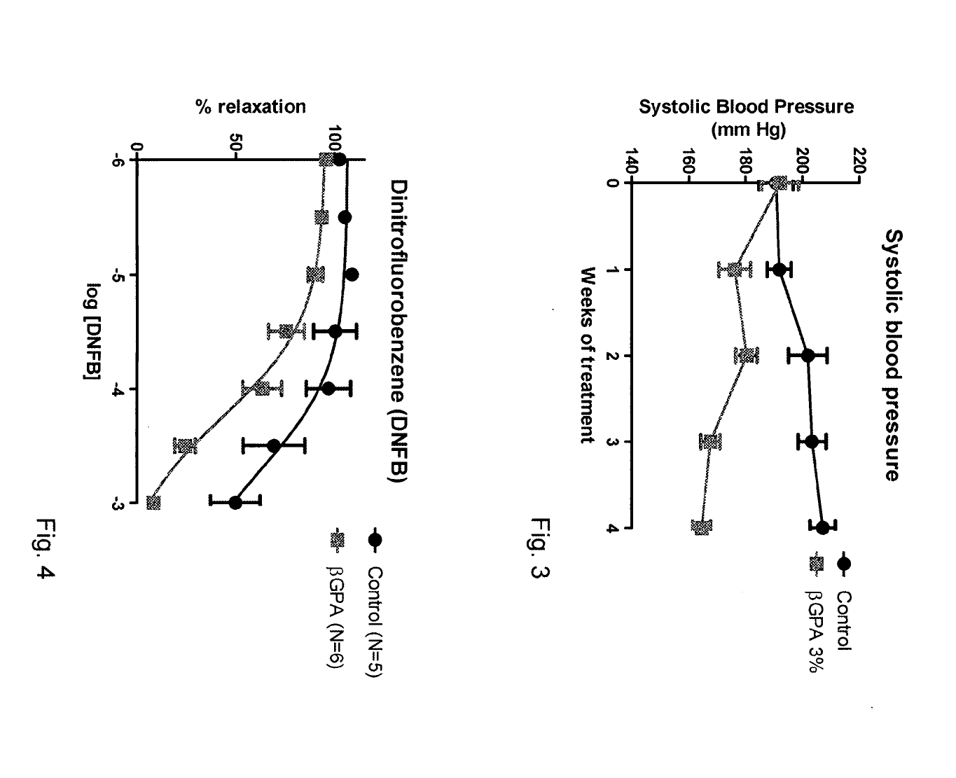

Treatment with β-GPA significantly reduced blood pressure compared to controls. See FIG. 3. Systolic pressure was reduced −42.7 (5.5) mm Hg, p<0.001; diastolic −35.3 (4.8) mm Hg, p<0.001. There were no significant weight changes and blood pressure reduction was independent of weight. Residual contractility after the CK inhibitor DNFB (10·3) was significantly lower in mesenteric arteries of rats treated with β-GPA compared to controls (see FIG. 4); −41.3 (13.0)% (p<0.01).

### Conclusion

To our knowledge, we show for the first time that CK inhibition reduces blood pressure in a non-obese, normoglycemic, high CK model, without hyperlipidaemia or other metabolic abnormalities. Modulation of the CK-system is therefore a possible target in the treatment of hypertension associated with high creatine kinase activity.

1. A method for the prevention and/or treatment of hypertension, asthma and/or coagulopathy in a subject with high creatine kinase activity, comprising administering to a subject with high creatine kinase activity a therapeutically effective amount of β-guanidinopropionic acid or a pharmaceutically acceptable salt thereof or a derivative thereof.

2. The method according to claim 1, wherein the β-guanidinopropionicacid is administered at a dose of 0.001 to 1000 mg per kg body weight.

3. The method according to claim 1, wherein the β-guanidinopropionic acid or a pharmaceutically acceptable salt thereof or a derivative thereof is administered at a daily dose in a range between 1 and 1000 mg per kg body weight, or in a range between 10 and 100 mg per kg body weight.

4. The method according to claim 1, wherein the β-guanidinopropionic acid is effectively taken up by a vascular cell, or a kidney and a vascular cell.

5. The method according to claim 1, wherein the hypertension or coagulopathy is characterized by high baseline creatine kinase plasma levels above 130 IU/L in adults.

6. A pharmaceutical composition comprising a therapeutically effective amount of β-guanidinopropionic acid or a pharmaceutically acceptable salt thereof or a derivative thereof, and a pharmaceutically acceptable carrier and/or an adjuvant, said adjuvant being selected from the group consisting of crystals, amorphous, liposomes, micelles, nanoparticles, microparticles, dendrimers, natural polymers, synthetic polymers, polysaccharides, lipids, deoxyribonucleic acids, ribonucleic acids, inorganic salts, organic salts, and surfactants.

7. The pharmaceutical composition according to claim 6, wherein the amount of β-guanidinopropionic acid or a pharmaceutically acceptable salt thereof or a derivative thereof is 0.01 to 100 wt %, 0.1 to 50 wt %, or 0.5 to 25 wt %.

8. The pharmaceutical composition according to claim 6 selected from the group consisting of a tablet, coated-tablet, effervescent tablet, soft-shell capsule, coated soft-shell capsule, hard-shell capsule, coated hard-shell capsule, solution, emulsion, suspension, suppository, aerosol, powder, granulate or lyophilized product.

9. The pharmaceutical composition according to claim 6, wherein the amount of β-guanidinopropionicacid or a pharmaceutically acceptable salt thereof or a derivative thereof in a single dosage form is between 0.1 and 1 gram.

10. The pharmaceutical composition according to claim 6, wherein the pharmaceutical composition is for administration orally, sublingually, parenterally, transcutaneously, intravenously, intramuscularly, intranasally, subcutaneously or via inhalation.

11. The pharmaceutical composition according to claim 6, wherein the pharmaceutical composition is administered 1 time per day, 2 times per day, or 3 times per day.

12. The pharmaceutical composition according to claim 7 comprising a further active ingredient as a hypertension medicament selected from the group consisting of ACE inhibitors, alpha blockers, angiotensin II receptor antagonists, beta blockers, calcium channel blockers and/or diuretics, angiotensinogenases and precursors thereof.

13. A method for the treatment and/or prophylaxis of hypertension, asthma and/or coagulopathy comprising administering to a subject in need thereof a therapeutically effective amount of the pharmaceutical composition as defined in claim 6.

14. A method of modulating at least one blood circulation parameter selected from blood pressure and blood platelet, aggregation in a subject, said method comprising interfering with the creatine kinase reaction by modulating the level of creatine, phosphocreatine and/or creatine kinase in a vascular smooth muscle cell, vascular endothelial cell or kidney cell in said subject, wherein said modulation is achieved by administering to a subject in need thereof a therapeutically effective amount of a pharmaceutical composition as defined in claim 6 or a substance that activates the creatine kinase reaction selected from an anabolic hormone including thyroxine hormone, a glucocorticoid hormone, phosphocreatine and creatine.

15. The method according to claim 14, wherein effective modulation of the creatine kinase reaction is indicated by a change in the level of creatine, phosphocreatine and/or creatine kinase in the serum of said subject.

16. The method according to claim 14, wherein said method of modulating a blood circulation parameter in a subject selected from blood pressure and blood platelet aggregation is a method for treating hypertension, coagulopathy and/or asthma in a subject with high creatine kinase activity, or a method for treating hypotension and/or thrombosis in a subject with low creatine kinase activity.

17. The method according to claim 16, wherein said method for treating hypertension and/or coagulopathy comprises the inhibition of the creatine kinase reaction by administering to a subject in need thereof a therapeutically effective amount of a pharmaceutical composition as defined in claim 7.

18. The method according to claim 16, wherein said method for treating hypotension and/or thrombosis comprises the activation of the creatine kinase reaction by administering to a subject in need thereof a therapeutically effective amount of a substance that activates the creatine kinase reaction selected from a anabolic hormone such as thyroxine hormone, a glucocorticoid hormone, creatine and phosphocreatine.
